(19) United States
(12) Patent Application Publication
(10) Pub. No.: US 2007/0044167 A1
(43) Pub. Date: Feb. 22, 2007

(54) Method for producing avian chimera using spermatogonial cells and avian chimera

(75) Inventors: Jae Yong Han, Seoul (KR); Jeong Mook Lim, Seoul (KR); Young Mok Lee, Kyunggi-do (KR); Jin Nam Kim, Kyunggi-do (KR); Yeong Ho Hong, Kyunggi-do (KR); Beom Ku Han, Seoul (KR)

Correspondence Address: CLARK & ELBING LLP, 101 FEDERAL STREET, BOSTON, MA, 02110, US

(73) Assignees: Avicore Biotechnology Institute Inc., Gunpo City (KR); Seoul National University Industry Foundation, Seoul (KR)

(21) Appl. No.: 10/567,815

(22) PCT Filed: Aug. 11, 2004
(86) PCT No.: PCT/KR04/02018

§ 371 Date: Apr. 10, 2006

(30) Foreign Application Priority Data
Aug. 11, 2003 (KR) 10-2003-0055326

(51) Int. Cl. A01K 67/027; C12N 5/06

(52) U.S. Cl. 800/19; 435/455; 435/349

(57) ABSTRACT

The present invention relates to a method for producing an avian chimera using spermatogonial cells which comprising the steps: (a) retrieving a testis from a donor ave; (b) isolating a testicular cell population from the testis; (c) culturing the testicular cell population in a medium supplemented with a cell growth factor to obtain a spermatogonial cell population; and (d) injecting the cultured spermatogonial cell population or testicular cell population into a testis of a recipient ave to produce the avian chimera. In addition, the present invention relates to a germline avian chimera.

### BACKGROUND OF THE INVENTION

1. Field of the Invention

The present invention relates to a method for producing an avian chimera using spermatogonial cells and methods for producing germline transmission avian chimeras and transgenic aves.

2. Description of the Related Art

In 1994, the transplantation of mouse spermatogonial cells was successfully carried out by microinjection (Brinster and Zimmerman, 1994; Brinster and Avarbock, 1994). Thereafter, many related reports and publications have been available. The earlier researches had been focused on the development of transplanting spermatogonial cells into a recipient seminiferous tubule (Ogawa et al., 1997; Nagano and Brinster, 1998; Nagano et al., 1998; Russell and Brinster, 1998; Russell et al., 1998).

According to the researches as to spermatogonia of Brinster's group published in 1994, spermatogonial cells derived from donor were successfully transplanted and finally produced sperms. Brinster and Zimmerman reported germline chimeras were produced by microinjection of heterogeneous mouse testicular cell mixture into the seminiferous tubule of a genetically sterile male mouse. Moreover, they verified that testicular cells containing lacZ as a marker gene may migrate into the basal membrane at the lowest portion of lumen in the seminiferous tubule.

The microinjection method had not been considered an efficient tool for the transplantation of spermatogonial cells. However, as being developed in the microinjection technique, it has been possible to fill fully the interior of the seminiferous tubule of the recipient with donor cells using the rete testis and the connection region of the seminiferous tubule.

Moreover, in 1994, Brinster and Avarbock shown that sperms harboring the lacZ gene were successfully produced in recipient testes by testicular cell transplantation. Moreover, they performed the sterilization of the recipient testis using chemicals and found that busulfan prevents spermatogenesis of the recipient, killing no endogenous spermatogonial cells. Therefore, it has been possible to enhance the transplantation efficiency by sterilizing recipients.

Thereafter, Jiang and Short (1995) reporting a method for transplanting spermatogonial cells have transplanted primordial germ cells and testicular cells developed and then examined their colonization in the testis of recipient mouse. As a result, it could be observed that the morphology of primordial germ cells was similar to those in the seminiferous tubule and germ cells isolated from postnatal testes underwent spermatogenesis by combination with testicular tissues of the recipient.

Clouthier et al (1996) have reported a xenografting method for spermatogonial cells, elucidating that spermatogenesis occurred from rat spermatogonial cells transplanted into Severe Combined Immune Deficiency (SCID) mouse and observing spermatozoa in mouse testes having the morphology of the head of rat spermatozoa which cannot be found in mouse spermatozoa. Previous researches and a common knowledge of one skilled in the art have said that sertoli cells might be closely related to the spermatogenesis, not supporting heterogeneous germ cells transplanted from other species. The success of the interspecies transplantation as discussed previously, provided a momentum of alteration in the conventional knowledge for the roles of sertoli cells. Therefore, it could be recognized that substances for spermatogenesis secreted from sertoli cells may serve as germ cells.

Although interspecies transplantation is successful in mice, other species exhibited no spermatogenesis following maturation of germ cells despite that immunodeficient mice were employed (Ogawa et al., 1996b; Dobrinski et al., 1999b). It may be realized that such failure in spermatogenesis is ascribed not to immune rejection response. In fact, it could be understood that the interspecies transplantation is very likely to occur only between species having close genetical and evolutionary relationship in consideration of the transplantation results between rat and mouse. In addition, for solving these problems described above, recent researches have been focused on the developmental timing of germ cells (Franca et al., 1998). The period of time required for maturing spermatozoa is at least 35 days in mouse and 52-53 days in rat. In this regard, under such circumstances, transplanting spermatogonial cells of mouse into testis of rat may be more effective.

Researches on the morphology of germ cells transplanted between different or same species have made under stereoscopic and electron microscopes (Russell and Brinster, 1996), observing that germ cells transplanted into the testis in mouse exhibited a deformed morphology. Theses observation results demonstrate that the spermatogenesis in rat is associated with sertoli cells of mouse.

Other researches on interspecies transplantation reported so far are as follows: transplantation of testicular cells of mouse, cow, monkey and human (Schlatt et al., 1999); transplantation of testicular cells of human into the testis of mouse (Zhang et al., 2003); transplantation of testicular cells of rabbit and dog into the testis of mouse (Dobrinski et al., 1999); and transplantation of testicular cells of livestock (cow, pig and horse) into the testis of mouse.

For employing the spermatogonial cells for novel transformation technology, pivotal technologies are the storage and in vitro culture of spermatogonial cells. It has been firstly reported that spermatogonial cells cultured in vitro for 3 months successfully were transplanted (Brinster and Nagano, 1996). Spermatogonial cells or a mixture of testicular cells cryopreserved in vitro before transplantation have been reported to be successfully transplanted (Avarbock et al., 1996).

The colonization of donor cells transplanted in the recipient testis has been observed (Nagano et al., 1999; Parreira et al., 1998). According to this report, the transplanted spermatogonial cells were observed on the bottom basal surface of the seminiferous tubule. Moreover, the paraffin section observation showed that spermatozoa derived from the donor cells transplanted were distributed at a ratio of 30% 3 months after the transplantation.

For the efficient transplantation, it is required to determine the optimum number of cells to be transplanted. The image analysis firstly suggested that the optimum number of cells for transplantation was 10⁷ donor cells/testis (Dobrinski et al., 1996b) and a low concentration of testosterone permitted a more efficient transplantation (Ogawa et al., 1998). While previous reports have not suggested, the subsequent publication reporting the transplantation using spermatogonial cells purified with antibody has suggested that testicular cells with 10-fold increased purity could be obtained and their transplantation leaded to the donor cell-originated population produced in the recipient (Shinohara et al., 1999).

Recently, the researches on transplantation of spermatogonial cells have been made in the subject of human (Schlatt et al., 1999). Spermatogonial cells isolated in his/her childhood may be used in adult life for transplantation for treating infertility and other disorders. Moreover, spermatozoa derived from spermatogonial cells have a potential for an artificial fertilization by in vitro culture and in vitro fertilization.

In an ave, the production of transgenic chicken and germline chimera has been already reported, using primordial germ cells or embryonic germ cells. Recently, a method for introducing genes into fertilized eggs by use of spermatozoa has been introduced (Qian et al., 2001). However, the method with very low efficiency also has many problems in establishing the transgenic systems since the stable gene transfer into individual is far poor and germline transmission is not verified.

However, spermatogonial cells may be easily obtained in massive manner from adult animals and have the potential to produce germline chimeras in a recipient testis, so that they may solve time-consuming and low efficiency problems associated with technologies using embryonic stem cells. Furthermore, the report on the spermatogenesis in a recipient testis by transplanting sepermatogonial cells with foreign gene (Nagano et al., 2000) demonstrates the breakthrough of transgenic animal production systems using spermatogonial cells.

Meanwhile, the production of avian chimera using spermatogonial cells has not been reported yet.

Throughout this application, various publications are referenced and citations are provided in parentheses. The disclosure of these publications in their entities are hereby incorporated by references into this application in order to more fully describe this invention and the state of the art to which this invention pertains.

### DETAILED DESCRIPTION OF THIS INVENTION

The present inventors have made intensive research to meet the long-felt need in the art. As a result, the present inventors have discovered that chimeras have been successfully produced using avian spermatogonial cells.

Accordingly, it is an object of the present invention to provide a method for producing an avian chimera using spermatogonial cells.

It is another object of the present invention to provide a germline transmission avian chimera.

It is still another object of the present invention to provide a method for producing a transgenic ave.

Other objects and advantages of the present invention will become apparent from the detailed description to follow taken in conjugation with the appended claims and drawings.

In one aspect of the invention, there is provided a method for producing an avian chimera using spermatogonial cells, which comprises the steps of: (a) retrieving a testis from a donor ave; (b) isolating a testicular cell population from the testis; (c) culturing the testicular cell population in a medium supplemented with a cell growth factor to obtain a spermatogonial cell population; and (d) injecting the cultured spermatogonial cell population or testicular cell population into a testis of a recipient ave to produce the avian chimera.

The present invention establishes avian chimera production systems using spermatogonial cells for the first time. The method of the present invention will be discussed with the descriptions of each step as follows:

Firstly, testes are retrieved from a donor ave. If the present invention is applied to a chicken, the age of a male chicken as a source of spermatogonial cells ranges, preferably from immediate time period after development to 70 weeks, more preferably, from immediate time period after development to 50 weeks, and most preferably, from 4 weeks to 30 weeks. The testes of chickens may be retrieved by isolating the cervical vertebra and dissecting.

Thereafter, a testicular cell population is isolated from the testis. The connective tissue and membrane around the testis isolated are removed and the tunica albuginea surrounding the testicular tissue is removed. The testis is then minced with using an anatomical knife and dissociated according to a variety of methods, thereby isolating testicular cells.

The term “testicular cell” used herein refers to a population of cells present in a testicular tissue, including spermatogonial stem cells; spermatogonial cells including all germ cells derived from spermatogonial stem cells; sertoli cells; Leydig cells and muscle cells associated with connective tissue. This term is used interchangeably with the term “testicular cell population”.

The testicular tissue may be dissociated according to various methods known to one skilled in the art. Preferably, the step of isolating testicular cells from testes is conducted by treating the testicular tissue retrieved with collagenase, trypsin or mixture thereof. More preferably, this step is conducted according to the 2-step enzymatic treatment, van Pelt method (1996) or collagenase-trypsin treatment as follows:

\textcircled{1} 2-Step Enzymatic Treatment

This method is conducted by the Ogawa et al (1997) method and its modified methods. The testicular tissue prepared is added into HBSS (Hank's Balanced Salt's solution) containing collagenase type I and incubated for a predetermined period of time, followed by the treatment with trypsin.

\textcircled{2} van Pelt Method (1996)

The testicular tissue prepared is dissociated in DMEM containing collagenase type I, trypsin, hyaluronidase II and DNase I.

\textcircled{3} Collagenase-Trypsin Treatment

The testicular tissue prepared is dissociated in HBSS containing collagenase type I and trypsin and then further dissociated by pipetting.

The testicular tissue lysate dissociated thus is then filtered thorough a suitable cell strainer (pore diameter about 70 μm) to collect testicular cells.

The testicular cells collected are cultured in a medium supplemented with a cell growth factor to obtain a spermatogonial cell population. The term “spermatogonial cell population” used herein refers to a population of cells consisting of spermatogonial cells to generate spermatocytes as well as a population of cells comprising not only spermatogonial cells but also a smaller population of spermatogonial stem cells and other testicular cells.

The medium useful in the culture of spermatogonial cells contains a cell growth factor as an essential ingredient, preferably, containing fibroblast growth factor (e.g., basic fibroblast growth factor), insulin-like growth factor-1, stem cell factor, glial derived neurotrophic factor or their combination, more preferably, containing fibroblast growth factor, insulin-like growth factor-1, stem cell factor or their combination, and most preferably, containing a mixture of fibroblast growth factor and insulin-like growth factor-1.

According to a preferred embodiment, the medium used in the culture of spermatogonial cells further contains differentiation inhibitory factor, most preferably, containing leukemia inhibitory factor. Accordingly, the most preferable combination of the growth factor and differentiation inhibitory factor is a mixture of fibroblast growth factor, insulin-like growth factor-1 and leukemia inhibitory factor.

In addition, it is preferred that the medium useful in the present invention contains avian serum (e.g., chicken serum), mammal serum (e.g., fetal bovine serum) or their combination. In addition to this, it is preferred that the medium contains antioxidant (e.g., β-mercaptoethanol), antibiotics-antimycotics, non-essential amino acids (e.g., arginin, asparagine, aspartic acid, glutamic acid, glycine, proline and serine), buffer (e.g., Hepes buffer) or their combination.

Although the culture of spermatogonial cells described previously may be performed with sertoli cells as feeder cells present in the testicular cell population, it is preferred that other types of cell are employed as feeder cells in the long-term culturing of spermatogonial cells. The feeder cell useful in the long-term culturing includes fibroblast, gonadal stroma cell, testicular stroma cell and mouse STO cell line (SIM mouse embryo-derived, Thioguanine- and Quabain-resistant fibroblast cell line), more preferably, including gonadal stroma cell and testicular stroma cell, and most preferably, including gonadal stroma cell. If the method of present invention is applied to chickens, it is preferred that the fibroblast, gonadal stroma cell and testicular stroma cell are chicken-derived. The feeder cells are deposited on the bottom layer of dishes or plates containing a medium and spermatogonial cells transferred to a medium attach on the feeder cell layer and proliferate.

The spermatogonial cell population cultured thus is injected into a testis of a recipient to produce a chimera. The spermatogonial cells to be injected are cultured as described above preferably for 5 days-4 months and more preferably for 5-30 days. The age of the male avian recipient ranges, preferably from immediate time period after development to 70 weeks, more preferably, from immediate time period after development to 50 weeks, and most preferably, from 4 days to 40 weeks.

The spermatogonial cell population cultured as well as the testicular cell population comprising spermatogonial cells can be directly used to produce a chimera.

The step of injecting spermatogonial cells or testicular cells into the testis is significantly important for the production of avian chimera. The injection may be preferably carried out by the injection into the seminiferous tubule, the injection into the epididymis or the injection into the rete testis, more preferably, by the injection into the seminiferous tubule of recipient, and most preferably, by the injection into the most upper part in the seminiferous tubule of a recipient.

According to a preferred embodiment, a testcross analysis is conducted after the step (d) to verify whether the recipient injected with the spermatogonial cell population is chimera or not. For example, where a donor is Korean Ogol chicken (i/i) having black feather and a recipient is White Leghorn (I/I) having white feather, the putative chimera produced by the procedures described above is crossbred with Korean Ogol chicken (i/i). If progenies having black feather are produced, the putative chimera can be identified as a genuine chimera.

The method of this invention can be used in any avian species, preferably, a chicken, a quail, a turkey, a duck, a goose, a pheasant or a pigeon, more preferably, a chicken.

The method of the present invention for producing a chimera may be conducted between different species as well as between the same species.

The present method aforementioned permits to produce germline chimera in more efficient and more convenient manner. Where a foreign gene is transferred into spermatogonial cells of a donor, the stable system for producing a transgenic ave can be provided.

In another aspect of this invention, there is provided an avian chimera characterized in that it maintains spermatogonial cells of a donor in its testis, it has the ability to produce spermatozoa from the spermatogonial cells and the spermatozoa undergo a germline transmission into progenies.

The avian chimera exhibiting the capacity of germline transmission of spermatogonial cells originated from a donor is firstly suggested by the present invention.

According to a preferred embodiment, the avian chimera of the present invention is produced by the present method described previously.

In still another aspect of this invention, there is provided a method for producing a transgenic ave, which comprises the steps of: (a) retrieving a testis from a donor ave; (b) isolating a testicular cell population from the testis; (c) culturing the testicular cell population in a medium supplemented with a cell growth factor to obtain a spermatogonial cell population; (c′) transferring a foreign gene into the spermatogonial cell population or testicular cell population; (d) injecting the spermatogonial cell population or testicular cell population into a testis of a recipient ave; and (e) producing a progeny from the recipient to obtain the transgenic ave.

In accordance with the present method, the transfer of a foreign gene into avian spermatogonial cells or testicular cells may be carried out by conventional gene transfer methods. For example, the method includes electroporation, liposome-mediated transformation (Wong et al., 1980) and retrovirus-mediated transformation (Chen et al., 1990; Kopchick et al., 1991; Lee & Shuman, 1990). It is preferred that the electroporation method is performed according to the procedures suggested by the present inventors (see, Korean Patent No. 305715).

According to a preferred embodiment, the foreign gene carries an antibiotic-resistance gene as a selection marker. It is preferred that the present method further comprises the step of selecting spermatogonial cells showing the antibiotic resistance property after step of (c), and the step of (d) is conducted using the antibiotic resistant spermatogonial cells. The selective marker useful in this invention may include any gene conferring antibiotic resistance to eukaryotic cells, for example, neomycin-, puromycin- and zeomycin-resistance genes.

It is preferred that the step of transplanting avian spermatogonial cells or testicular cells into the testis of the recipient is carried out by microinjecting spermatogonial stem cells into the seminiferous tubules.

The recipient is mated with other individual to generate progenies, finally obtaining a transgenic ave harboring the foreign gene.

### BRIEF DESCRIPTION OF THE DRAWINGS

FIG. 1 schematically represents a process for producing an avian chimera using spermatogonial cells according to the preferred embodiment of this invention.

FIG. 2 is a photograph demonstrating the possibility in the injection of spermatogonial cells into the testis of chicken. It is observed that the inner portion of the seminiferous tubule of testis is stained with trypan blue.

FIG. 3 is a photograph showing progenies of germline chimeras produced according to the present method.

FIG. 4 is a photograph showing the morphology of spermatogonial cells of 4 weeks aged Korean Ogol chicken depending on the duration of in vitro culture. The number in photographs denotes the duration (day) of in vitro culture.

FIG. 5 is a photograph showing the morphology of spermatogonial cells of 24 weeks aged Korean Ogol chicken depending on the duration of in vitro culture. The number in photographs denotes the duration (day) of in vitro culture.

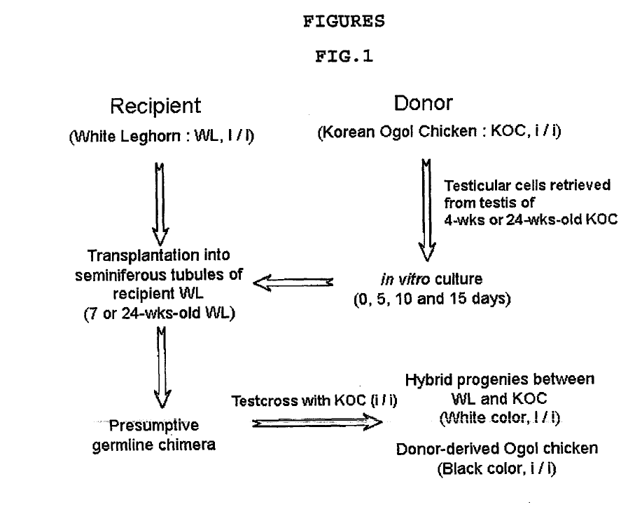

### EXAMPLES

The following specific examples are intended to be illustrative of the invention and should not be construed as limiting the scope of the invention as defined by appended claims.

Experiment Animals

Korean Ogol chicken (KOC) and White Leghorn (WL) were used as experimental animals. Each animal was used to isolate donor cells from testes, which were transplanted into the recipient testes.

Isolation of Donor Spermatogonial Cells

Donor spermatogonial cells were isolated from testes of 4 or 24 weeks-aged donor KOC according to the two-step enzymatic method of Brinster et al. (1994) with a little modification based on the features of chicken testes.

Being different from other mammals, chicken testis is present within the abdominal cavity and symmetrically attaches to the dorsal region of the abdominal cavity near kidney. The left testis is generally larger than the right testis and they were surrounded by the abdominal air sac with hanging on the dorsal region. Therefore, testes were retrieved from experimental animals by anesthesia and surgery. 10-20 testes (4 weeks age) and 2 testes (24 weeks age) were retrieved for use in each experiment and rapidly transferred into the phosphate buffered saline (PBS) solution. After retrieving testes, the connective tissue and membrane around testicular tissue were removed and the tunica albuginea surrounding testicular tissue was removed using a micro-forceps. The testes were minced with a dissecting knife under a stereomicroscope and were immersed in HBSS (Hank's Balanced Salt's solution, Invitrogen) containing collagenase type I (1 mg/ml, Sigma) to incubate for 15 min in 37° C. shaking incubator. The collagenase treated testes were then washed with HBSS and treated with 0.25% trypsin-1 mM EDTA for 15 min. Testicular tissues dissociated thus were filtered through 70 μm cell strainer (Falcon 2350), finally measuring the survival rate and number of spermatogonial cells using trypan blue dye.

In vitro Culture of Spermatogonial Cells

After disassociating spermatogonial cells into single cell, spermatogonial cells were in vitro cultured for a short period of time before transplantation. The durations for culturing were 0, 5, 10 and 15 days. Spermatogonial cells isolated from young testes (4 weeks age) and sexual maturated testes (24 weeks age) were used for culturing. 1×10⁸ spermatogonial cells seeded on 100 mm cell culture dish were incubated at 37° C in 5% CO₂ incubator.

The media for spermatogonial cell culture were formulated by incorporating into DMEM (Dulbecco's minimal essential medium, Gibco Invitrogen) 10% (v/v) fetal bovine serum for ES cell use only (FBS, Hyclone, Logan UT), 1× antibiotics-antimycotics (Invitrogen), 2% chicken serum, 10 mM non-essential amino acids, 10 mM Hepes buffer, 0.55 mM β-mercaptoethanol, and a mixture of 10 ng/ml human leukemia inhibitory factor (Sigma), 10 ng/ml human basic fibroblast growth factor (Sigma) and 100 ng/ml human insulin-like growth factor-I (Sigma) as growth factors. The seeded cells were cultured in the incubator for 5, 10 and 15 days, respectively. The cells cultured were then dissociated by an enzymatic treatment using 0.25% trypsin-1 mM EDTA for 5 min, followed by centrifugation to enrich cells for transplantation.

Transplantation of Spermatogonial Cells

Isolated or in vitro cultured spermatogonial cells were centrifuged and enriched to 2×10⁷ cells/50-100 μl and then transplanted into the testis of recipient chicken. Transplantation was carried out in adult (24 weeks) and young (7 weeks) recipient chickens, respectively. For a general anesthesia, 10 mg/kg (20 μl) of ketamin anesthesia (YUHAN Corporation) were injected via a wing vein. The right-lower abdominal of the chicken anesthetized was dissected and the existence of the testis present in the subvertebral was observed. The cell suspension prepared previously was injected into the testis using a syringe adapted with 33 G needle (Hamilton, 100 μl). For injection, the end of the injection needle was located at the outer membrane of testis, so that the cells were transplanted into the most upstream portion of the seminiferous tubule. Following the injection, the incised intimal and adventitia of abdominal were sutured using a surgical suture needle and thread. The surgery region was disinfected and administered with antibiotics.

Observation of Spermatogonial Cell Injection

In anatomical structure of chickens, testis is located at the subvertebral region in abdomen; therefore, it is difficult to carry out the surgery for exposing testis under a microscope in chickens as the general transplantation in mice. Therefore, the thickness of injection needles and angle of injection becomes significantly important to successfully carry out cell transplantation in chickens. In this regard, it was confirmed whether the cells could be actually introduced into the seminiferous tubule by injecting the trypan blue dye into the isolated testes using the syringe needle having the same gage as that for transplantation of cells under a microscope.

Testcross Analysis for Identification of Germline Chimera

Testcross analysis was undertaken to identify the spermatogeneis of spermatogonial cells of KOC transplanted into the testis of WL recipient. The feather color of KOC is black because of the recessive pigmentation gene (i/i) and that of WL is white due to the dominant pigmentation inhibitory gene (I/I), so that white progenies (I/i) hatches after mating WL with KOC. However, black KOC progenies (i/i) hatches after mating ovum of KOC with spermatozoa originated from spermatogonial cells transplanted into the recipient testes, which enables progenies to be identified as a germline chimera.

### RESULTS

Confirmation of Spermatogonial Cell Transplantation

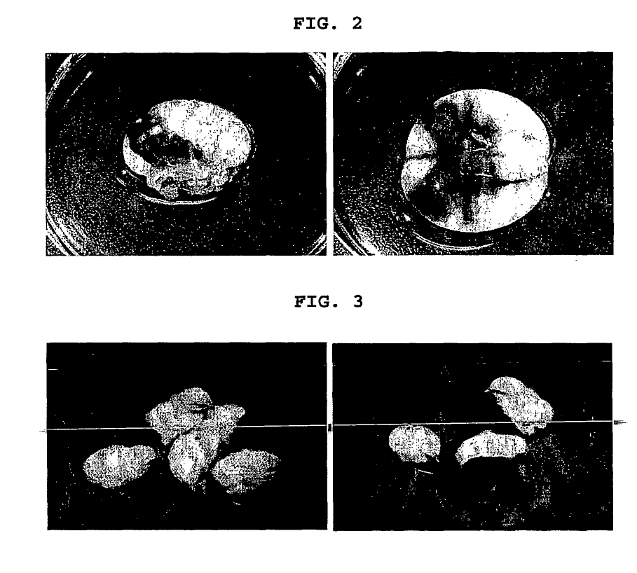

In anatomical structure of chickens, testis is located at the subvertebral region in abdomen; therefore, it is difficult to carry out the surgery for exposing testis under a microscope in chickens as the general transplantation in mice. Therefore, the thickness of injection needles and angle of injection becomes significantly important to successfully carry out cell transplantation in chickens. In this regard, it was confirmed whether the cells could be actually introduced into the seminiferous tubule by injecting the trypan blue dye into the isolated testes using the syringe needle having the same gage as that for transplantation of cells under a microscope. As shown in FIG. 2, it was observed that the trypan blue solution was introduced through the seminiferous tubule and dispersed throughout the testis. Therefore, it would be understood that spermatogonial cells could be successfully transplanted into recipient testes according to the surgery method using suitable needles found in the present invention.

Comparision of Production Efficiency of Germline Chimera

Testcross analysis were undertaken to establish the conditions for producing germline chimera. Two weeks after recovering from transplantation, transplanted 4 chickens per each experiment were crossbred with female KOC. Spermatogonial cells in vitro cultured for 5-10 days were used for the testcross analysis.

As found in Table 1 and FIG. 2, germline chimeras were produced by the transplantation of spermatogonial cells. However, the production efficiency was lower than that of mouse. It was suggested that such lower production efficiency is due to the competition of transplanted spermatogonial cells with endogenous recipient-originating spermatoginial cells. It would be appreciated that the lower production efficiency could be overcome by using sterilization technologies such as the treatment of busulphan.

TABLE 1
Comparison of production efficiency of germline chimera
depending on age of donor and recipient

| Systems of germline | | | | No. (%) of |
|---|---|---|---|---|
| chimera production | | | No. of | chickens |
| | Age of | No. of | chickens | proved as |
| Age of donor | recipient | transplanted | undertaken | germline |
| chicken | chicken | chickens | testcross | chimeras |
| Adult | Adult | 16 | 16 | 3 (18.8) |
| (24 wks) | (24 wks) | | | |
| Young (4 | Adult | 16 | 16 | 0 (0.0) |
| wks) | (24 wks) | | | |
| Adult | Young (7 wks) | 16 | 16 | 1 (6.3) |
| (24 wks) | | | | |
| Young (4 | Young (7 wks) | 16 | 16 | 1 (6.3) |
| wks) | | | | |

TABLE 2
Comparison of germline transmission efficiency of individual
germline chimera depending on age of donor and recipient

| | | | No. (%)^{a} | |
|---|---|---|---|---|
| Systems of germline chimera | | | of progenies | |
| production | | | produced | No. (%)^{b} of |
| | Age of | ID No. of | from | progenies |
| Age of donor | recipient | germline | the germline | with white |
| chicken | chicken | chimears | chimera | feather |
| Adult (24 wks) | Adult (24 wks) | YM13 | 2 (3.6) | 54 (96.4) |
| | | YM14 | 1 (3.2) | 31 (96.8) |
| | | YM38 | 2 (1.6) | 124 (98.4) |
| Young (4 wks) | Adult (24 wks) | — | — | — |
| Adult (24 wks) | Young (7 wks) | YM75 | 1 (0.9) | 11 (99.1) |
| Young (4 wks) | Young (7 wks) | YM72 | 1 (4.8) | 20 (95.2) |

^{a}Percentage of the number of donor cell-derived progenies(black feather) from germline chimeric chickens undertaken testcross analysis.
^{b}Percentage of the number of hybrid chickens between WL and KOC.

Meanwhile, white progenies hatched from normal WL and KOC, and black progenies hatched from WL male injected with spermatogonial cells of KOC, as shown in FIG. 3. These results indicate that injected spermatogonial cells of KOC normally divide and differentiate in the testis of WL recipient.

In vitro Culture of Spermatogonial Cells

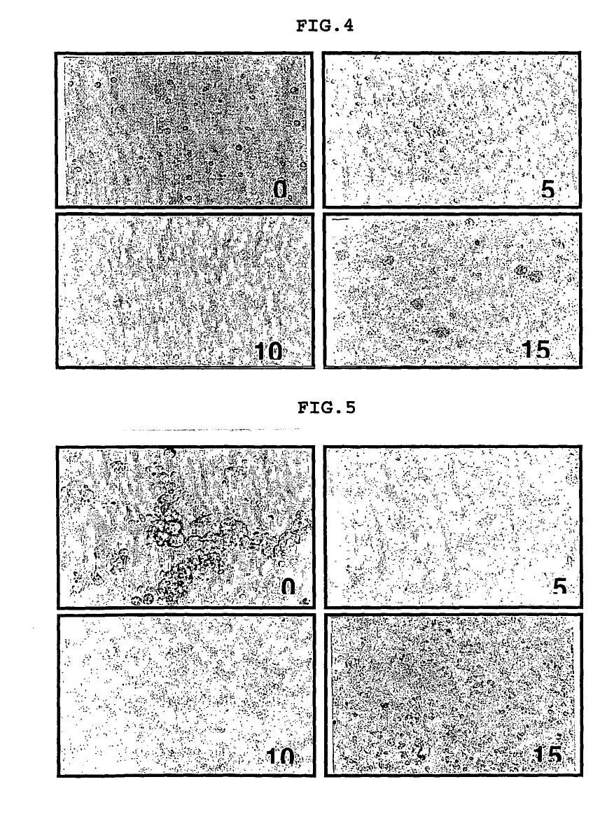

In case of spermatogonial cells of short-term in vitro cultured KOC, the cells could be maintained by in vitro culture for 15 days. As represented in FIGS. 4 and 5, 4 weeks aged testicular cells in vitro cultured were stably maintained and colonized after 15 days to show the increased cell number. In addition, 24 weeks aged adult testicular cells in vitro cultured were stably maintained and colonized after 15 days to show the increased cell number, similar to the results of the culture of 4 weeks aged cells.

The production efficiency of germline chimera was compared following the short-term in vitro culture. As shown in Tables 3 and 4, germline chimeras were produced by the transplantation of spermatogonial cells in vitro cultured for 5 days and 10 days. Moreover, the production efficiency of progeny, i.e., the germline transmission efficiency was revealed to be the highest in the transplantation of spermatogonial cells in vitro cultured for 5 days.

TABLE 3
Comparison of production efficiency of germline chimera
depending on duration of in vitro culture

| Systems of germline | | | | |
|---|---|---|---|---|
| chimera production | | | | No. (%) of |
| Duration | | | No. of | chickens |
| of in | No. of | No. of | chickens | proved as |
| vitro | transplanted | transplanted | undertaken | germline |
| culture | cells | chickens | testcross | chimeras |
| 0 day | 2.0 × 10⁷ | 16 | 16 | 2 (12.5) |
| 5 days | 2.0 × 10⁷ | 16 | 16 | 2 (12.5) |
| 10 days | 2.0 × 10⁷ | 16 | 16 | 1 (6.3) |

TABLE 4
Comparison of germline transmission efficiency of individual
germline chimera depending duration of in vitro culture

| Systems of germline | | | | |
|---|---|---|---|---|
| chimera production | | | No. (%)^{a} of | |
| Duration | | | progenies | No. (%)^{b} of |
| of in | No. of | ID No. of | produced from | progenies |
| vitro | transplanted | germline | the germline | with white |
| culture | cells | chimeras | chimera | feather |
| 0 day | 2.0 × 10⁷ | YM14 | 1 (3.2) | 31 (96.8) |
| | | YM38 | 2 (1.6) | 124 (98.4) |
| 5 days | 2.0 × 10⁷ | YM13 | 2 (3.6) | 54 (96.4) |
| | | YM72 | 1 (4.8) | 20 (95.2) |
| 10 days | 2.0 × 10⁷ | YM75 | 1 (0.9) | 111 (99.1) |

^{a}Percentage of the number of donor cell-derived progenies(black feather) from germline chimeric chickens undertaken testcross analysis.
^{b}Percentage of the number of hybrid chickens between WL and KOC.

### INDUSTRIAL APPLICABILITY

As described previously, the present invention provides a method for producing an avian chimera using spermatogonial cells and a method for producing a germline transmission avian chimera and transgenic ave. According to the method of the present invention, a germline avian chimera could be conveniently prepared with improved efficiency.

Having described a preferred embodiment of the present invention, it is to be understood that variants and modifications thereof falling within the spirit of the invention may become apparent to those skilled in this art, and the scope of this invention is to be determined by appended claims and their equivalents.

### REFERENCES

1. Avarbock, M. R., et al., 1996. Reconstitution of spermatogenesis from frozen spermatogonial stem cells. Nat. Med. 2, 693-696.
2. Brinster, R. L., et al., 1994. Germline transmission of donor haplotype following spermatogonial transplantation. Proc. Natl. Acad. Sci. 91, 11303-11307.
3. Brinster, R. L., et al., 1998. Spermatogonial transplantation, cryopreservation and culture. Cell Dev. Biol. 9, 401-409.
4. Brinster, R. L., et al., 1994. Spermatogenesis following male germ-cell transplantation. Proc. Natl. Acad. Sci. 91, 11298-11302.
5. Cibelli, J. B., et al., 1998. Cloned transgenic calves produced from nonquiescent fetal fibroblasts. Science. 280, 1256.
6. Clouthier, D. E., et al., 1996. Rat spermatogenesis in mouse testes following spermatogonial stem cell transplantation. Nature. 381, 418-421.
7. Dobrinski, I., et al., 1999a. Transplantation of germ cells from rabbits and dogs into mouse testes. Biol. Reprod., 61, 1331-1339.
8. Dobrinski, I., et al., 1999b. Computer assisted image analysis to assess colonization of recipient seminiferous tubules by spermatogonial stem cells from transgenic donor mice. Mol. Reprod. Dev. 53, 142-148.
10. Dobrinski, I., et al., 2000. Germ cell transplantation from large domestic animals into mouse testes. Mol. Reprod. Dev. 57, 270-279.
9. Dym, M., 1994. Spermatogonial stem cells of the testes. Proc. Natl. Acad. Sci. 91, 11287-11289.
10. Franc a, L., et al., 1998. Germ cell genotype controls cell cycle during spermatogenesis. Biol. Reprod. 59, 1371-1377.
11. Jiang, F.-U., et al., 1998. Different fate of primordial germ cells and gonocytes following transplantation. APMIS. 106, 53-63.
12. Jiang, F.-X., et al., 1995. Male germ cell transplantation in rats: apparent synchronization of spermatogenesis between host and donor seminiferous epithelia. Int. J. Androl. 18, 326-330.
13. Nagano, M., et al., 1998. Spermatogonial transplantation and reconstitution of donor cell spermatogenesis in recipient males. APMIS. 106, 47-57.
14. Nagano, M., et al., 1998. Culture of mouse spermatogonial stem cells. Tissue Cell 30, 389-397.
15. Nagano, M., et al., 1999. Pattern and kinetics of mouse donor spematogonial stem cell colonization in recipient testes. Biol. Reprod. 60, 1429-1436.
16. Ogawa, T., et al., 1997. Transplantation of testes germinal cells into mouse seminiferous tubules. Int. J. Dev. Biol. 41, 111-121.
17. Ogawa, T., et al., 1998. Leuprolide, a gonadotropin-releasing hormone agonist, enhances colonization after spermatogonial transplantation into mouse testes. Tissue Cell. 30, 583-588.
18. Ogawa, T., et al., 1999a. Recipient preparation is critical for spermatogonial transplantation in the rat. Tissue Cell, 31, 461-472.
19. Ogawa, T., et al., 1999b. Xenogeneic spermatogenesis following transplantation of hamster germ cells to mouse testes. Biol. Reprod. 60, 515-521.
20. Parreira, G., et al., 1998. Development of testes cell transplants. Biol. Reprod. 59, 1360-1370.
21. Parreira, G. G., et al., 1999. Development of germ cell transplants: morphometric and ultrastructural studies. Tissue Cell. 31, 241-254.
22. Russell, L. D., et al., 1996. Ultrastructural observations of spermatogenesis following transplantation of rat testes cells into mouse seminiferous tubules. J. Androl. 17, 615-627.
23. Schlatt, S., et al., 1999. Germ cell transfer into rat, bovine, monkey, and human testes. Hum. Reprod. 14, 144-150.
24. Schnieke, A. E., et al., 1998. Human factor IX transgenic sheep produced by transfer of nucli from transfecterd fetal fibroblast. Science. 278, 2130-2133
25. Shinohara, T., et al., 1999. B1- and A6-integrin are surface markers on mouse spermatogonial stem cells. Proc. Natl. Acad. Sci. 96, 5504-5509.
26. Van Pelt A M, et al., 1996 Isolation of the synchronized A spermatogonial cells from adult vitamin A-deficient rat testes. Biol Reprod. 55:439-444
27. Vincent, S., et al., 1998. Development (Cambridge, U.K.) 125: 4585-4593.
28. Wilmut, I., et al., 1997. Viable offspring derived from fetal and adult mammalian cells. Nature. 385: 810-813
29. Yoshinaga K, et al., 1991. Role of c-kit in mouse spermatogenesis: identification of spermatogonial cells as a specific site of c-kit expression and function. Development. 113: 689-699
30. Zhang Z, et al., 2003. Successful intra- and intrespecific male germ cell transplantation in the rat. Biol. Reprod. 68: 961-967
31. Zirkin B R, et al., 1994. Is FSH required for adult spermatogenesis? J Androl. 15:273-276

What is claimed is:

1. A method for producing an avian chimera using spermatogonial cells, which comprises the steps of:
(a) retrieving a testis from a donor ave;
(b) isolating a testicular cell population from said testis;
(c) culturing said testicular cell population in a medium supplemented with a cell growth factor to obtain a spermatogonial cell population; and
(d) injecting said cultured spermatogonial cell population or said testicular cell population into a testis of a recipient ave to produce said avian chimera.

2. The method according to claim 1, wherein said step (b) is conducted by treating a tissue of said testis retrieved with collagenase, trypsin or mixture thereof.

3. The method according to claim 1, wherein said cell growth factor is selected from the group consisting of fibroblast growth factor, insulin-like growth factor-1, stem cell factor and combination thereof.

4. The method according to claim 1, wherein said medium further comprises differentiation inhibitory factor.

5. The method according to claim 1, wherein said differentiation inhibitory factor is leukemia inhibitory factor.

6. The method according to claim 1, wherein said medium contains a supplement comprising a mixture of fibroblast growth factor, insulin-like growth factor-1 and leukemia inhibitory factor.

7. The method according to claim 1, wherein said medium further comprises a serum and an antioxidant.

8. The method according to claim 1, wherein said step (d) is conducted by injecting said cultured spermatogonial cell population or said testicular cell population into the seminiferous tubule of said recipient.

9. The method according to claim 8, wherein said step (d) is conducted by injecting said cultured spermatogonial cell population or said testicular cell population into the most upper portion of the seminiferous tubule of said recipient.

10. The method according to claim 1, wherein said ave is selected from the group consisting of a chicken, a quail, a turkey, a duck, a goose, a pheasant or a pigeon.

11. The method according to claim 1, wherein said donor and said recipient are different species.

12. The method according to claim 1, wherein said method further after the step (d) comprises the step of conducting a testcross to verify whether said recipient injected with said cultured spermatigonial cell population is chimera.

13. An avian chimera characterized in that it maintains spermatogonial cells of a donor in its testis, it has the ability to produce spermatozoa from said spermatogonial cells and said spermatozoa undergo a germline transmission into progenies.

14. The avian chimera according to claim 13, wherein said avian chimera is produced by any one of the methods of claims 1 to 11.

15. A method for producing a transgenic ave, which comprises the steps of:
(a) retrieving a testis from a donor ave;
(b) isolating a testicular cell population from said testis;
(c) culturing said testicular cell population in a medium supplemented with a cell growth factor to obtain a spermatogonial cell population;
(c′) transferring a foreign gene into said cultured spermatogonial cell population or testicular cell population;
(d) injecting said cultured spermatogonial cell population or testicular cell population into a testis of a recipient ave; and
(e) producing a progeny from said recipient to obtain said transgenic ave.
